(19) United States
(12) Patent Application Publication
(10) Pub. No.: US 2005/0055061 A1
(43) Pub. Date: Mar. 10, 2005

(54) Cardiac implant device

(76) Inventor: Asher Holzer, Halfa (IL)

Correspondence Address: DR. MARK FRIEDMAN LTD.;C/o Bill Polkinghorn, Discovery Dispatch, 9003 Florin Way, Upper Marlboro, MD, 20772, US

(21) Appl. No.: 10/656,172

(22) Filed: Sep. 8, 2003

(51) Int. Cl. A61N 1/362

(52) U.S. Cl. 607/35

(57) ABSTRACT

A heart implant device for associating with a heart of a living body, the device including: (a) a housing for securely associating with heart tissue, the housing encompassing a space, the housing including a conductive coil, and (b) a ferromagnetic element disposed within the space, the element for moving relative to the coil so as to produce electrical energy within the living body.

### FIELD AND BACKGROUND OF THE INVENTION

The present invention relates to a micro-generator implant device for providing power within a living body, the device being securely associated with heart tissue.

Many implantable medical devices, such as pacemakers and defibrillators, require an electrical energy source. In pacemakers and defibrillators, this energy source normally is provided by a battery pack that is contained within the implanted device. Although rechargeable batteries have been successfully employed in a variety of applications, some present day pacemakers and defibrillators use non-rechargeable batteries.

Surgery, with its attendant risks, discomforts, and cost is required when it becomes necessary to replace an implanted medical device. Because the batteries are hermetically sealed within the implanted device, the entire medical device must be surgically replaced if the batteries become depleted. To avoid or postpone surgery, it thus would be beneficial to provide longer lasting implantable devices. Longer life for an implant can be achieved by using a larger battery, however, this undesirably increases the size of the implant.

Despite the prominence of non-rechargeable batteries for powering implanted medical devices, some situations favor the use of rechargeable batteries. Some implanted medical devices, such as ventricular assist devices, require large amounts of electrical power to operate. Such devices often are powered by an external, non-implanted power source with direct electrical connection through the skin to the implant or indirectly via induction coils. It is often desirable, however, to detach the external power source from the implant, for example, when the patient bathes. During the time that the external power source is detached, the implanted device operates from battery power. Because of the large energy demand of some such implanted devices, it would be desirable to provide a rechargeable battery source for the implant to avoid having to surgically intervene to replace the non-rechargeable batteries once they become depleted. Upon reconnecting the external power source, the internal rechargeable battery pack could be recharged.

In applications in which rechargeable batteries are employed, a system to recharge the batteries is necessary. Such a recharging system should be non-invasive or minimally invasive. Several recharging techniques, and the inherent deficiencies thereof, are surveyed in U.S. patent application Ser. No. 10/266,681 to Holzer.

U.S. patent application Ser. No. 10/266,681, which is incorporated by reference for all purposes as if fully set forth herein, teaches a device for generating power within a living body. Various sources of internal mechanical energy can be harnessed by the device, including motion of heart muscle tissue, motion of blood passing through a blood vessel, motion of a limb, and/or motion of the entire body.

Various embodiments of the device utilize the twisting motion of the heart and/or the displacement occurring due to the contraction and expansion of the heart. For utilization of the twisting motion of the heart, an implant having a ferromagnetic shaft and coil arrangement is implanted in the body in an orientation that enables the shaft to move with respect to the surrounding in response to the twisting motion resulting from each heartbeat. The magnetic field that is created induces an AC electrical current that is harnessed to supply power for the implant or for another device within the body, as needed.

Similarly, the displacement resulting from the contraction and expansion of the heart is utilized by implanting a micro-generator near the heart, preferably oriented with the axis of the shaft disposed in a substantially perpendicular fashion with respect to the heart, such that with each heartbeat, the shaft moves back and forth in relation to the coil.

The micro-generator devices taught by U.S. patent application Ser. No. 10/266,681 answer the need for powering a wide variety of devices for implanting within a living body. However, certain specific embodiments require affixation of the device to heart tissue. The anchoring of the device to heart tissue is extremely problematic. The above-described motions of the heart place various pressures on the device, pressures of a large magnitude that develop rapidly during the course of each heartbeat. Moreover, the anchoring mechanism must be robust enough to withstand these motions and pressures over the requisite lifetime of the device, which is typically several years at the very least. There is, therefore, a recognized need for, and it would be highly advantageous to have, a device for and method of robustly securing an implanted medical device to heart tissue. It would be of further advantage to have a device that is easy to implant, reduces risk and discomfort to the patient, is inexpensive to manufacture, and is substantially maintenance-free.

### SUMMARY OF THE INVENTION

The present invention is a heart implant device for associating with a heart of a living body, the device including: (a) a housing for securely associating with heart tissue, the housing encompassing a space, the housing including a conductive coil, and (b) a ferromagnetic element disposed within the space, the element for moving relative to the coil so as to produce electrical energy within the living body.

According to further features in the described preferred embodiments, the housing is for securely juxtaposing with the heart tissue.

According to still further features in the described preferred embodiments, the housing is for attaching directly to the heart tissue, preferably by a fixture selected from the group of fixtures including a staple, a suture, and a tie.

According to still further features in the described preferred embodiments, the housing is for disposing generally around a circumference of the heart.

According to still further features in the described preferred embodiments, the housing is for enveloping the heart by at least 60 degrees, more preferably by at least 120-180 degrees, and most preferably, by at least 240 degrees.

According to still further features in the described preferred embodiments, the housing is a ring for substantially encompassing the heart.

According to still further features in the described preferred embodiments, the housing is shaped to spiral around the heart.

According to still further features in the described preferred embodiments, the housing is for securely associating with an epicardium.

According to still further features in the described preferred embodiments, the housing is for securely associating within a pericardium.

According to still further features in the described preferred embodiments, a first end of the housing is disposed within a second end of the housing.

According to still further features in the described preferred embodiments, the first end includes the ferromagnetic element.

According to still further features in the described preferred embodiments, the housing is attached to the heart tissue near a first end of the housing, such that a second end of the housing has at least one degree of freedom to move in response to movement of the heart tissue.

According to still further features in the described preferred embodiments, the housing includes a plurality of compartments, each compartment including a ferromagnetic element.

According to still further features in the described preferred embodiments, each of the compartments further includes a spring mechanism for returning the ferromagnetic element from a wall of the compartment.

According to still further features in the described preferred embodiments, the housing includes a flexible joint for absorbing stress due to a movement of the heart tissue.

According to still further features in the described preferred embodiments, the flexible joint includes a bellowed section.

According to still further features in the described preferred embodiments, the conductive coil is disposed externally to the housing.

According to still further features in the described preferred embodiments, the conductive coil is disposed within the housing.

According to still further features in the described preferred embodiments, the ferromagnetic element is a shaft.

According to still further features in the described preferred embodiments, the ferromagnetic element is a ball.

According to still further features in the described preferred embodiments, the housing further includes a biocompatible external layer for contacting the heart tissue.

According to still further features in the described preferred embodiments, the housing further includes a biocompatible layer disposed to physically and electrically isolate the heart tissue from the coil.

According to still further features in the described preferred embodiments, the external wall of the housing flares out so as to provide increased surface area for improving a distribution of pressure applied to the heart tissue.

According to still further features in the described preferred embodiments, the external wall of the housing flares out so as to provide increased surface area for securing the housing to the heart tissue.

According to still further features in the described preferred embodiments, a first end of the housing is disposed externally to the heart.

According to still further features in the described preferred embodiments, the first end includes a compartment, the compartment including the ferromagnetic element.

According to still further features in the described preferred embodiments, the heart implant further includes: (c) a pacemaking element for stimulating contractions of muscle tissue in the heart.

According to still further features in the described preferred embodiments, the device is designed and configured for anchoring between the myocardium and epicardium.

According to still further features in the described preferred embodiments, the device is designed and configured for anchoring within a pericardium encompassing the heart.

According to still further features in the described preferred embodiments, the device is designed and configured for anchoring between the pericardium and epicardium.

According to still further features in the described preferred embodiments, the device is designed and configured for anchoring within a coronary sinus.

According to still further features in the described preferred embodiments, disposed within the space within the housing is a spring mechanism for returning the ferromagnetic element from the wall of the housing.

According to another aspect of the present invention there is provided a method for associating a heart implant device with a heart of a living body, the method including the steps of: (a) providing a device including: (i) a housing for securely associating with heart tissue, the housing encompassing a space, the housing including a conductive coil, and (ii) a ferromagnetic element disposed within the space, the element for moving relative to the coil so as to produce electrical energy within the living body, and (b) attaching the device to the heart tissue.

According to another aspect of the present invention there is provided a heart implant device for associating with a heart of a living body, the device including: (a) a housing for securely associating with heart tissue of the heart; (b) a conductive coil, and (c) a ferromagnetic element, wherein either the coil or the ferromagnetic element is securely associated with the housing, and wherein the coil and the ferromagnetic element are designed and configured for moving relative to one another in response to a movement of the heart tissue, so as to produce electrical energy within the living body.

### BRIEF DESCRIPTION OF THE DRAWINGS

The invention is herein described, by way of example only, with reference to the accompanying drawings. With specific reference now to the drawings in detail, it is stressed that the particulars shown are by way of example and for purposes of illustrative discussion of the preferred embodiments of the present invention only, and are presented in the cause of providing what is believed to be the most useful and readily understood description of the principles and conceptual aspects of the invention. In this regard, no attempt is made to show structural details of the invention in more detail than is necessary for a fundamental understanding of the invention, the description taken with the drawings making apparent to those skilled in the art how the several forms of the invention may be embodied in practice.

In the drawings:

FIG. 1a is a schematic diagram of a human heart;

FIG. 1b is a cross-sectional view of the heart of FIG. 1a, taken along A-A;

FIG. 2a is a schematic illustration of a hollow, generally ring-shaped micro-generator device, according to one embodiment of the present invention;

FIG. 2b is a schematic illustration of the device of FIG. 2a, affixed to epicardial tissue;

FIG. 3 is a schematic illustration of a hollow, generally spiral-shaped micro-generator device disposed between the pericardium and the myocardium, and encompassing a heart, according to another embodiment of the present invention;

FIG. 4a is a schematic illustration of a hollow, ring-shaped micro-generator device having bellowed joints, according to another embodiment of the present invention;

FIG. 4b is a schematic illustration of a hollow, generally ring-shaped micro-generator device having a narrow tail end disposed within a wide head end thereof, according to another embodiment of the present invention;

FIG. 5 is a schematic illustration of an inventive, hollow, generally ring-shaped micro-generator device having multiple compartments, each compartment for independent generation of energy;

FIG. 6 is a schematic illustration of a generally arc-shaped micro-generator in which a first end of the housing is secured to heart tissue, and a second end of the housing has at least one degree of freedom to move in response to movement of the heart tissue, according to another embodiment of the present invention;

FIG. 7 is a schematic illustration of a cross-section of an inventive micro-generator having a flared sidewall for distributing pressures resulting from movement of the heart tissue;

FIG. 8 is a schematic illustration of an internally-powered pacemaker system, and

FIG. 9 is a schematic illustration of an internally-powered pacemaker system disposed between the myocardium and the epicardium.

### DESCRIPTION OF THE PREFERRED EMBODIMENTS

The present invention is an anchored micro-generator implant for providing power within a living body, and more particularly, for providing power to an implant in the proximity of the heart or to the heart itself.

The principles and operation of the anchored micro-generator implant of the present invention may be better understood with reference to the drawings and the accompanying description.

Before explaining at least one embodiment of the invention in detail, it is to be understood that the invention is not limited in its application to the details of construction and the arrangement of the components set forth in the following description or illustrated in the drawing. The invention is capable of other embodiments or of being practiced or carried out in various ways. Also, it is to be understood that the phraseology and terminology employed herein is for the purpose of description and should not be regarded as limiting.

As used herein in the specification and in the claims section that follows, the term “implant” refers to any powered device implanted in the body, including, but not limited to, pacemakers, defibrillators, internal communication devices, monitoring devices such as: heart condition, heart beat, ECG, electrocardiogram, blood contents, blood pressure, temperature, blood leak from a graft stent, blood vessel ruptures, and combinations thereof. The term “implant” is meant to include intra-cardiac and intra-coronary devices. The implant may be incorporated as a part of a coronary stent, blood vessel stent, etc., and most preferably to a pacemaker. The term “implant” is meant to specifically include various internally implanted devices that are traditionally powered by external energy sources or by batteries and other energy storage devices.

As used herein in the specification and in the claims section that follows, the term “heart tissue” is specifically meant to include the epicardium, which contacts the surface of the heart, and more generally, the pericardium surrounding the heart.

As used herein in the specification and in the claims section that follows, the term “ferromagnetic” refers to any uncharged material capable of attracting others. The term also includes materials that have the capability to be magnetized. The term “ferromagnetic” is specifically meant to include materials possessing paramagnetic, ferromagnetic, and superparamagnetic properties.

As used herein in the specification and in the claims section that follows, the term “spring mechanism” includes any of various varieties of springs, spring-loaded plates, bumpers, and pre-tensioned projections.

As used herein in the specification and in the claims section that follows, the term “biocompatible material” refers to a material that does not produce a toxic, injurous, or immunological response in living tissue.

As used herein in the specification and in the claims section that follows, the term “ball”, used within a conductive coil, is meant to include various oval-shaped objects designed for moving relative to the housing of the coil.

As used herein in the specification and in the claims section that follows, the term “shaft”, used within a conductive coil, is specifically meant to include various curved or arc-shaped objects designed for moving relative to the housing of the coil, and particularly, within curved, tube-shaped housings.

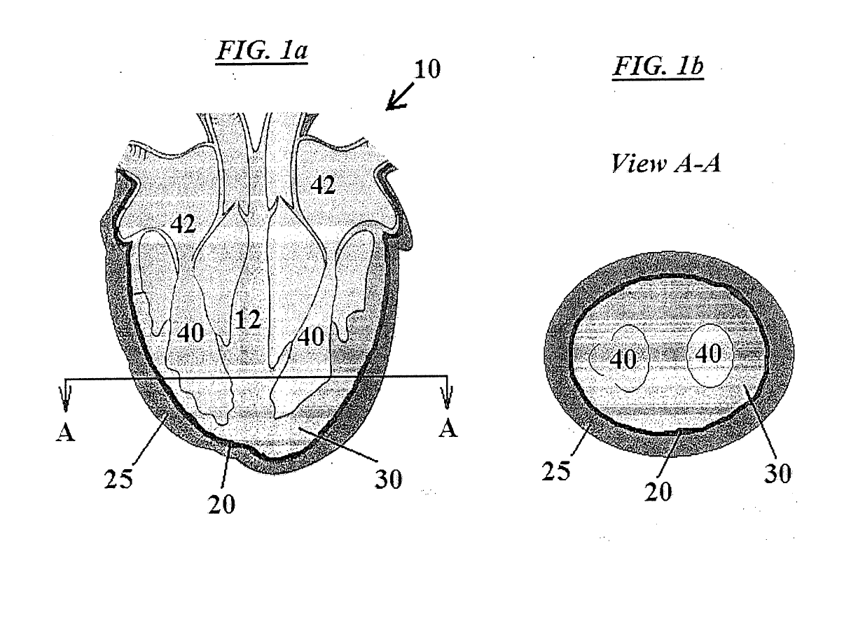

A human heart 10 is illustrated in FIG. 1a. Heart 10 contains four chambers, divided vertically by a membrane, or septum 12. Each side of heart 10 has two chambers—an atrium above 42 and a ventricle 40 below. Blood is pumped out of one side of heart 10, and through the lungs, before being introduced to the other side of heart 10. No blood passes across septum 12.

The blood is moved physically by the contractions of the heart muscle, or myocardium 30, which envelopes the heart chambers. The outermost layer enveloping heart 10 is the pericardium 25, a loose protective sac that is flexible enough to allow heart 10 to expand and contract during the pumping cycle. The inner layer of this sac, the epicardium 20, adheres closely to the surface of heart 10.

A schematic, cross-sectional view of the heart of FIG. 1a, taken along A-A, is provided in FIG. 1b. Myocardium 30 is seen to encompass the heart chambers, e.g., ventricle 40. Myocardium 30 is, in turn, enveloped by epicardium 20 and by pericardium 25.

The micro-generator generates electricity by means of a ferromagnetic material moving relative to a coil, as disclosed in U.S. patent application Ser. No. 10/266,681 to Holzer.

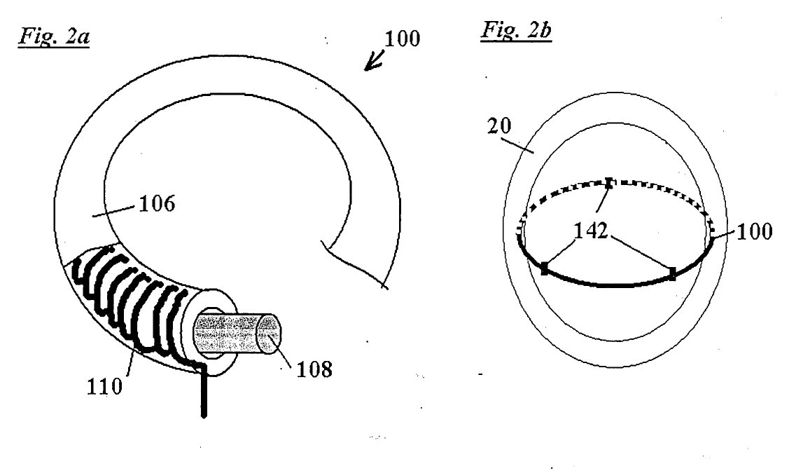

A schematic illustration of a hollow, generally ring-shaped micro-generator device is provided in FIG. 2a. Micro-generator 100 includes a hollow, generally cylindrical housing 106 having a moving (e.g., sliding) ferromagnetic shaft 108 disposed therein. Affixed to or within a wall of housing 106 is a conductive coil 110.

According to a first embodiment of the present invention, housing 106 is designed and configured as a ring structure for substantially encompassing the heart (myocardium). Preferably, micro-generator 100 is inserted between the pericardium and the heart, where cleavage planes exist. These cleavage planes are an eminently suitable place for micro-generator 100, which is squeezed and held in place there (e.g, between the epicardium and the myocardium, or within the pericardium). Another preferred location for micro-generator 100 is within the coronary sinus (not shown). The micro-generator 100 is placed in an orientation (see FIG. 2b) that enables shaft 108 to move back and forth within housing 106, powered by the beating and twisting motions of the heart. One preferred method of fixing micro-generator 100 to epicardium 20 utilizes staples or stitches (sutures) 142.

Shaft 108 is made of any of various ferromagnetic materials, such as iron, nickel or alloys thereof having the requisite magnetic properties. The outer surface of housing 106, which contacts living tissue, is preferably made of various biocompatible materials that are known in the art.

It must be emphasized that structural modification of epicardial tissue is very common in modern heart surgery. Several examples are provided hereinbelow:
cardiomyostimulator implantation: the latissimus dorsi muscle is cut off and replanted around the heart, enabling for epicardial pacing leads to be inserted into the right ventricle and the subsequent epicardial tunneling and pocket creation for the cardiomyostimulator.
off-pump coronary artery bypass surgery: the epicardial tissues are cut adjacent to a vessel in order to construct the distal anastomosis.
“waffle” operation: the epicardial tissue is cut in multiple longitudinal and transverse directions, thereby protecting the myocardium and the coronary arteries.
transmyocardial revascularization: channels are cut in the epicardium in order to introduce a fiber into the left ventricle.
myocardial patching: a myocardial patch is sutured to the heart by anchoring the sutures within tunnels cut in the epicardial tissues.

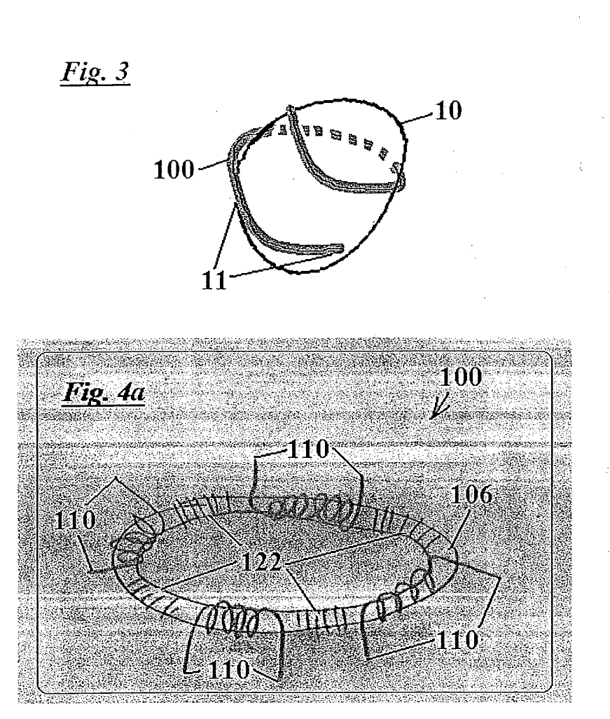

FIG. 3 is a schematic illustration of a hollow, generally spiral-shaped micro-generator device 100 disposed between the epicardium and the pericardium and encompassing heart 10, according to another embodiment of the present invention. Alternatively, micro-generator device 100 encompasses a portion of heart 10, as represented by segment 11. Segment 11 is preferably designed to encompass at least 60 degrees of heart 10, and most preferably at least 240 degrees. In some cases, more than a full 360 degrees, and even at least 420 degrees, is warranted.

The at least partial encompassing of heart 10 provides a large area for affixing device 100 to heart 10, and perhaps more importantly, allows device 100 to grip heart 10, such that various pressures resulting from the movement of heart 10 are absorbed and distributed along the length of device 100. The secure association with heart 10 also ensures that the mechanical energies associated with the multi-dimensional motion of heart 10 are more efficiently absorbed and utilized by micro-generator device 100. Device 100 may have a tube shape, and may be either solid or flexible. Device 100 may contain flexible and compressible sections to allow following the dynamic shape of the heart.

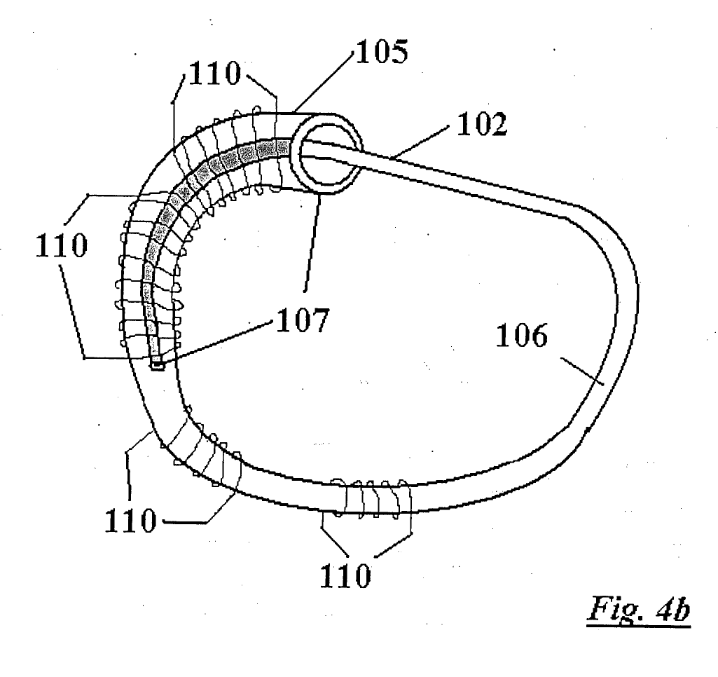

In another preferred embodiment, provided in FIG. 4a, housing 106 of micro-generator 100 has flexible joints or bellows 122 for imparting longitudinal flexibility to housing 106 and for absorbing stresses and pressures (“strain-release”) caused by the natural movements of the heart.

In yet another preferred embodiment, shown schematically in FIG. 4b, hollow and generally ring-shaped housing 106 has a narrow tail end 102 designed and configured for disposing within a wide head end 105 of housing 106, so as to enable a “tail-in-head” configuration around the heart. During expansion of the heart, a portion of tail end 102 is forced out of head end 105. Subsequently, during contraction of the heart, tail end 102 penetrates more deeply into head end 105. Hence, the “tail-in-head” configuration serves to absorb and distribute various stresses and pressures caused by the natural movements of the heart.

Various positionings of coil 110 along the length of housing 106 are possible. As described hereinabove, a moving ferromagnetic element (not shown), such as a shaft, ball, etc., is disposed within housing 106. In one preferred embodiment, conductive coil 110 is disposed near head end 105. Tail end 102 contains a ferromagnetic material, such that throughout the contraction and expansion of the heart, the motion of tail end 102 with respect to the overlapping portion 107 of head end 105 produces electrical energy. In this embodiment, tail end 102 essentially functions as a moving ferromagnetic shaft, obviating the need for an additional moving ferromagnetic element.

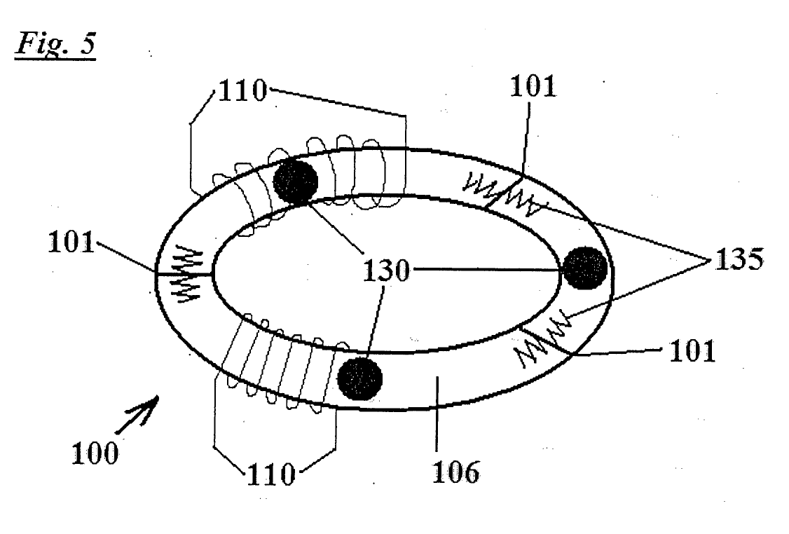

FIG. 5 is a schematic illustration of another embodiment of the present invention, in which housing 106 of a ring-shaped micro-generator 100 is divided into a plurality of compartments, each compartment designed to independently generate energy. The length of each of the three longitudinal compartments is defined by partitions 101. A ferromagnetic ball, or cylindrical shaft or bar 130 is disposed within each compartment, for moving relative to coils 110. Coils 110 are preferably disposed within or around housing 106.

During each heartbeat, the displacement and twisting of the heart shake and deform micro-generator 100, causing each ball 130 to roll or slide along the length of housing 106, within its respective compartment, so as to induce electricity.

Preferably, bumpers 135 are disposed at each end of the compartments, to enhance the motion of balls 130, and to reduce the probability of a ball 130 sticking to an end of the compartment.

The micro-generator 100 shown in FIG. 5 has three stand-alone systems working in parallel, each system designed to provide the requisite power for the implant device, such that even if one or two systems fail over time, for whatever reason, micro-generator 100 continues to provide sufficient power. This is especially important for implanted systems, which must be robustly and reliably designed to operate over many years without fail.

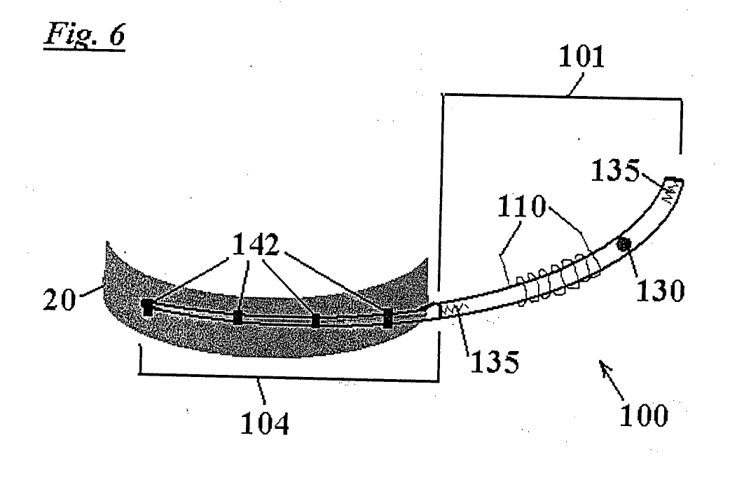

According to another embodiment of the present invention is schematically illustrated in FIG. 6. A generally arc-shaped micro-generator 100 has a first end 104 of the housing for securing to heart tissue, and a second end having at least one degree of freedom to move in response to movement of the heart tissue.

By way of example, first end 104 of micro-generator 100 is inserted underneath pericardium 25, and anchored at points 142 to the heart (myocardium) by sutures or staples. Compartment 101, containing a ferromagnetic ball 130, is disposed within the free end of micro-generator 100, and is encompassed by one or more conductive coils 110. During movements of the heart, compartment 101 is flung, causing ball 130 to travel longitudinally therein so as to produce electricity. The ends of compartment 101 are equipped with bumpers 135, as elaborated hereinabove. Micro-generator 100 is advantageously positioned in such a way that compartment 101 is on the outside of the pericardium, and may be moved back and forth inside a tube located there. In such a configuration a hole in the pericardium is needed. Moving parts are enclosed by the tube to avoid friction with living tissue.

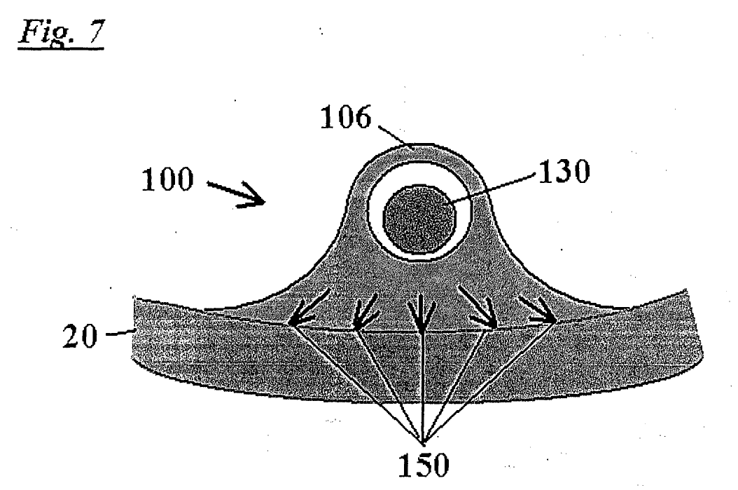

In another embodiment of the present invention, a schematic cross-section of which is provided in FIG. 7, a housing 106 of micro-generator 100 is equipped with a flared sidewall 146 for distributing pressures 150 resulting from movement of the heart tissue. Since, the available area for distributing these pressures is greatly increased, the pressures exerted on the pericardium 25, per unit area, are reduced. Hence the stress placed on any area having a suture, staple, or other affixing means, is correspondingly lowered, such that the connection between micro-generator 100 and epicardium 20 (or other heart tissue) is more robust. Optionally or additionally, flared sidewall 146 can be oriented in the direction of myocardium 30, so as to reduce the pressure (or “footprint”) exerted on the myocardium.

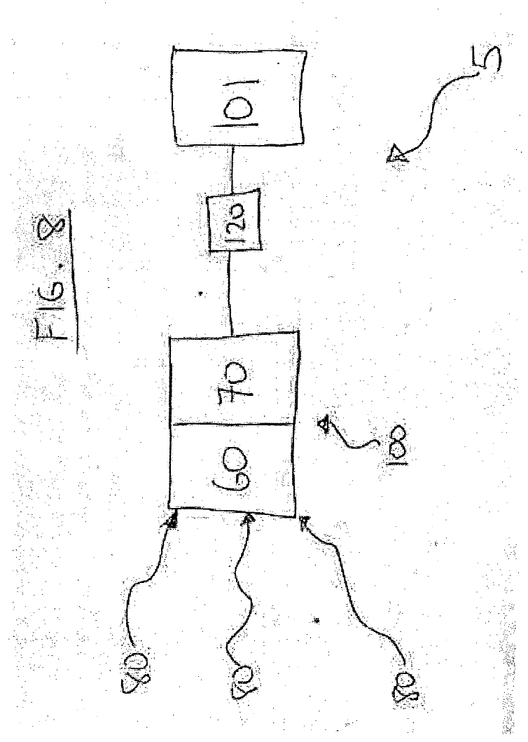

FIG. 8 is a schematic illustration of an internally-powered pacemaker system 5 including a micro-generator 100 and a pacemaking unit 101 operatively attached thereto, via energy storage unit 120. A heart motion 80 is harnessed by micro-generator 100 so as to charge energy storage unit 120, which in turn powers pace maker 101.

Micro-generator 100 includes a mechanical section 60 for harnessing the mechanical energy from a natural body movement, and a conversion section 70 in which mechanical energy from mechanical section 60 is converted to electrical energy.

Energy storage unit 120 is preferably selected from a wide variety of known internal energy storage units, including, but not limited to, capacitors and rechargeable batteries.

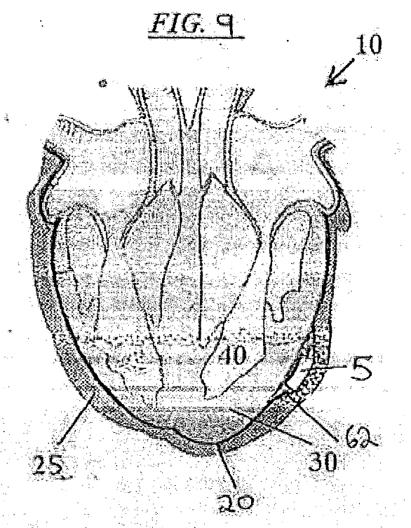

FIG. 9 is a schematic illustration of internally-powered pacemaker system 5 disposed between myocardium 30 and epicardium 20, along a cleavage plane 62.

Internally-powered pacemaker system 5 enables pace maker 101 (see FIG. 8) to pace heart 10 from a position outside of myocardium 30. This obviates the need for replacing an expended battery, the need for a lead wire, and the need for puncturing the myocardium with the lead wire (to secure the lead) and introducing the lead wire into the chambers (ventricle 40 and/or atrium) of the heart.

In another preferred embodiment, internally-powered pacemaker system 5 is disposed within pericardium 25. In yet another preferred embodiment, internally-powered pacemaker system 5 is disposed within the coronary sinus (not shown).

The insertion of the inventive device into the coronary sinus can be accomplished by one skilled in the art, using known procedures. At present, most of the devices associated with craniological treatments like coronary stents, pacemakers, and internal defibrillator devices are introduced to the heart via the blood vessels, and the introduction of the inventive device into the coronary sinus involves no additional technological hurdles.

The insertion of the inventive device into the pericardium 25 can also be accomplished by one skilled in the art, using other known procedures, many of which are related to laparoscopy. During the past few years, minimally invasive procedures based on laparoscopy and the like have been introduced to the medical community. These kinds of procedures are characterized by fast recovery, shorter hospitalization time, and low morbidity. Such procedures are being used in the removal of gall bladder stones, treating hernias, and various gynecological procedures.

The suggested method makes use of the wide experience already accumulated in laparoscopic procedures, and can make use of some laparoscopes already in the market. The method of inserting the micro-generator device and other devices associated therewith is preferably performed as follows:
1. A standard laparoscope with a viewing channel, an optical channel (for bringing light inside), and a working channel is inserted in the body in proximity to the heart.
2. A punch in the pericardium is performed through the working channel, using standard techniques.
3. The cleavage plane between the myocardium and the pericardium (or epicardium) is revealed.
4. A balloon is inserted to expand the cleavage plane.
5. The inventive device is inserted via the working channel.
6. The above steps should be performed under vision, using the laparoscope viewing channel, and may be confirmed by other means, including x-rays, ultra-sound and other modalities.
7. Anchoring the device to the heart tissue is done through the working channel of the laparoscope.
   It should be emphasized that in order to push an arc-shaped or curved implant device, a flexible laparoscope, or a modified laparoscope should be used, so as to allow a smooth deployment through the working channel.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims. All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention.

1. A heart implant device for associating with a heart of a living body, the device comprising:
(a) a housing for securely associating with heart tissue, said housing encompassing a space, said housing including a conductive coil, and
(b) a ferromagnetic element disposed within said space, said element for moving relative to said coil so as to produce electrical energy within the living body.

2. The implant device of claim 1, wherein said housing is for securely juxtaposing with said heart tissue.

3. The implant device of claim 1, wherein said housing is for attaching directly to said heart tissue.

4. The heart implant device of claim 3, wherein said housing is for attaching directly to said heart tissue by a fixture selected from the group of fixtures including a staple, a suture, and a tie.

5. The heart implant device of claim 1, wherein said housing is for disposing generally around a circumference of the heart.

6. The heart implant device of claim 1, wherein said housing is for enveloping the heart by at least 180 degrees.

7. The heart implant device of claim 6, wherein said housing is a ring for substantially encompassing said heart.

8. The heart implant device of claim 1, wherein said housing is shaped to spiral around the heart.

9. The heart implant device of claim 1, wherein said housing is for securely associating with an epicardium.

10. The heart implant device of claim 1, wherein said housing is for securely associating within a pericardium.

11. The heart implant device of claim 7, wherein a first end of said housing is disposed within a second end of said housing.

12. The heart implant device of claim 11, wherein said first end includes said ferromagnetic element.

13. The heart implant device of claim 1, wherein said housing is attached to said heart tissue near a first end of said housing, such that a second end of said housing has at least one degree of freedom to move in response to movement of said heart tissue.

14. The heart implant device of claim 1, wherein said housing includes a plurality of compartments, each compartment of said compartments including said ferromagnetic element.

15. The heart implant device of claim 14, wherein each of said compartments further includes a spring mechanism for returning said ferromagnetic element from a wall of said compartment.

16. The heart implant device of claim 1, wherein said housing includes a flexible joint for absorbing stress due to a movement of said heart tissue.

17. The heart implant device of claim 16, wherein said flexible joint includes a bellowed section.

18. The heart implant device of claim 1, wherein said conductive coil is disposed externally to said housing.

19. The heart implant device of claim 1, wherein said conductive coil is disposed within said housing.

20. The heart implant device of claim 1, wherein said ferromagnetic element is a shaft.

21. The heart implant device of claim 1, wherein said ferromagnetic element is a ball.

22. The heart implant device of claim 1, wherein said housing further includes a biocompatible external layer for contacting said heart tissue.

23. The heart implant device of claim 1, wherein said housing further includes a biocompatible layer disposed to physically and electrically isolate said heart tissue from said coil.

24. The heart implant device of claim 1, wherein an external wall of said housing flares out so as to provide increased surface area for improving a distribution of pressure applied to said heart tissue.

25. The heart implant device of claim 1, wherein an external wall of said housing flares out so as to provide increased surface area for securing said housing to said heart tissue.

26. The heart implant device of claim 1, wherein a first end of said housing is disposed externally to the heart.

27. The heart implant device of claim 26, wherein said first end includes a compartment, said compartment including said ferromagnetic element.

28. The heart implant device of claim 1, further comprising:
(c) a pacemaking element for stimulating contractions of muscle tissue in the heart.

29. The heart implant device of claim 28, wherein the device is designed and configured for anchoring between a myocardium and epicardium of the heart.

30. The heart implant device of claim 28, wherein the device is designed and configured for anchoring within a pericardium encompassing the heart.

31. The heart implant device of claim 28, wherein the device is designed and configured for anchoring within a coronary sinus.

32. The heart implant device of claim 1, wherein the device is designed and configured for anchoring between a myocardium and epicardium of the heart.

33. The heart implant device of claim 1, wherein the device is designed and configured for anchoring within a pericardium encompassing the heart.

34. The heart implant device of claim 1, wherein the device is designed and configured for anchoring within a coronary sinus.

35. The heart implant device of claim 1, wherein disposed within said space is a spring mechanism for returning said ferromagnetic element from a wall of said housing.

36. A method for associating a heart implant device with a heart of a living body, the method comprising the steps of:
(a) providing a device including:
(i) a housing for securely associating with heart tissue, said housing encompassing a space, said housing including a conductive coil, and
(ii) a ferromagnetic element disposed within said space, said element for moving relative to said coil so as to produce electrical energy within the living body, and
(b) attaching the device to said heart tissue.

37. A heart implant device for associating with a heart of a living body, the device comprising:
(a) a housing for securely associating with heart tissue of the heart;
(b) a conductive coil, and
(c) a ferromagnetic element,
wherein one of said coil and said ferromagnetic element is securely associated with said housing, and wherein said coil and said ferromagnetic element are designed and configured for moving relative to one another in response to a movement of said heart tissue, so as to produce electrical energy within the living body.
